(19) United States
(12) Patent Application Publication
(10) Pub. No.: US 2002/0038147 A1
(43) Pub. Date: Mar. 28, 2002

(54) Breast implant

(76) Inventor: Archibald S. Miller III, Tulsa, OK (US)

Correspondence Address: HEAD, JOHNSON & KACHIGIAN, 228 W 17TH PLACE, TULSA, OK, 74119, US

(21) Appl. No.: 09/999,747

(22) Filed: Oct. 31, 2001

Related U.S. Application Data
(63) Continuation-in-part of application No. 09/425,352, filed on Oct. 22, 1999.

(51) Int. Cl. A61F 2/12

(52) U.S. Cl. 623/8; 623/901

(57) ABSTRACT

An improved permanently implantable breast tissue prosthesis comprising angularly and immutably attached base and dome envelopes wherein the base envelope is of a substantially triangular shape and the dome envelope is of a substantially discoid shape, each envelope having a shell defining an inner fluid containable chamber and an outer textured surface to be in direct contact with breast tissue and a valve formed as a part of a wall in base and dome envelopes, the valve facilitating the introduction, containment or removal of fluid within the containable chamber of each envelope.

### REFERENCE TO PENDING APPLICATIONS

This application is a continuation-in-part of U.S. Ser. No. 09/425,352, filed Oct. 22, 1999 entitled “Breast Implant”.

### REFERENCE TO MICROFICHE APPENDIX

This application is not referenced in any microfiche appendix.

### TECHNICAL FIELD OF THE INVENTION

This invention relates in general to the field of implantable breast prosthesis, and more particularly to a breast prostheses which presents a “life-like” or “ptotic” shape attributable to angularly attached base and dome envelopes.

### BACKGROUND OF THE INVENTION

Implantable breast prostheses are used as augmentation implants to modify the size and/or shape of an intact breast; and as reconstructive implants to restructure or reform an area in which a breast has been previously removed or modified. Early attempts at breast modification called for material to be injected directly into the breast parenchyma. Such early attempts were not successful as injected material would disperse or could not be contoured to provide for a normal breast “shaping”. Later attempts used discrete implants, usually a single chamber implant filled with a silicone product (See U.S. Pat. Nos. 3,665,520; 3,681,787; 4,455,691; 4,472,226; 4,573,999; 4,772,284). These later attempts often did not present a normal contour to a modified breast, and they required relatively large incisions for insertion. Other implants present contoured or irregular shape, but usually have a single chamber, and when suspended in the human breast tend to deform with muscle contracture and scar formation.

Yet other prostheses have used multiple chambers (See U.S. Pat. No. 4,507,810 with multiple intercommunicating chambers). These implants often fail to yield a normal contour to the reconstructed or augmented breast, cannot be adjusted or “customized” for the individual recipient, and typically require large incisions.

Recently, separate “stacked” implants have been utilized in reconstruction or augmentation procedures. These implants generally consist of two or more implants stacked upon each other as required in the mammoplasty process to achieve projection while providing a conical shape, peaking at the nipple-areolar complex. However, there has been a problem with this type of implant procedure as “stacked” implants tend to drift apart or separate. Also, they may rotate/shift in relation to one another, in relation to the breast, and they may not remain in an eccentric position relative to each other.

An additional problem with implants of the contemporary art lies in the attachment means to connect envelopes of multi-envelope breast implant structures. Typically, such attachment means rely upon tape or connective tab like structures to attach envelopes to one another. Difficulty arises when independent shifting of any one envelope places stress upon the attachment means causing the attachment means to precipitate a rupture or tear in the attaching or attached envelope, thus causing fluid previously contained therein to leak out of the envelope or envelopes a sack like breast cavity created to accommodate the implantable breast prosthesis.

The present invention overcomes the problems described above by providing an improved breast prosthesis comprised of two immutably and angularly attached envelopes with continuously textured external textured services which allow the secure positioning and life like appearance of a female human breast. The base or lower envelope of the instant invention is substantially triangular in form and allows for an angular attachment by a chemical, heat, or other bonding means to a dome envelope which is generally discoid in structure. Once bonded together, the prosthesis of the instant invention is inserted into a cavity specifically formed for implantation purposes and each envelope is filled with a fluid substance such as but not limited to saline, gel, or other like fluid well known to those skilled in the art to achieve a desired breast form. The textured surface of the instant invention allows for “gripping” abutting breast tissue where upon said prosthesis remains fixed in its position following closure of incisions intentionally made for implantation purposes.

### BRIEF SUMMARY OF THE INVENTION

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

In this respect, before explaining at least one embodiment of the invention in detail, it is to be understood that the invention is not limited in this application to the details of construction and to the arrangement so the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore that the claims be regarded as including such equivalent constructions insofar as they do not depart from the spirit and scope of the present invention.

Further, the purpose of the foregoing abstract is to enable the U.S. Patent and Trademark Office and the public generally, and especially the design engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is an object of the instant invention to provided a prosthesis in which a dome envelope and a base envelope are immutably attached at an angle, thus allowing unequal expansion of fluid to yield a human breast shape.

It is also an object of the instant invention to provide an implantable prosthesis which embodies a textured backing which is continuous with base and dome envelopes form and texture.

It is a further object of this invention to provide an implant with envelopes of substantially discoid and triangular shapes attached to each other at an angle.

It is another object of this invention to provide an implant with a textured surface for positioning and securing the implant to the breast tissues so that it will not turn or slip.

It is an additional object of this invention to profile an implant with two, or more, separate implant elements attached to each other.

It is yet another object of this invention to provide an implant in which each implant element is independently and variably fillable with liquid or other filling material.

It is another object of this invention to provide an implant that may be inserted into the breast through a relatively small incision.

A further object is to provide an implant that will impart an essentially normal contour to the human breast after implantation.

Another object is to provide an implant that will provide a “normal” texture and feel to the breast after it is implanted.

It is yet a further object of this invention to provide an implant that may be individualized or customized by the surgeon for each patient.

It is also an object of this invention to provide a method for making implants and for using implants in the human breast.

These objects are meant to be illustrative and not limiting. The manner of operations, novel features and further objectives and advantages of this invention maybe better understood by reference to the accompanying drawings, description and claims.

### DESCRIPTION OF THE DRAWINGS

FIG. 1 is front view of the two element breast implant.

FIG. 2 is a side elevational view of the instant invention.

FIG. 3 is a top elevational view of the instant invention.

FIG. 4 is a bottom elevational view of the instant invention.

FIG. 5 is a cross-sectional view of the instant invention.

### DETAILED DESCRIPTION OF THE PREFERRED EMBODIMENT

While the making and using of various embodiments of the present invention are discussed in detail below, it should be appreciated that the present invention provides for inventive concepts capable of being embodied in a variety of specific contexts. The specific embodiments discussed herein are merely illustrative of specific manners in which to make and use the invention and are not to be interpreted as limiting the scope of the instant invention.

The claims and the specification describe the invention presented and the terms that are employed in the claims draw their meaning from the use of such terms in the specification. The same terms employed in the prior art may be broader in meaning than specifically employed herein. Whenever there is a question between the broader definition of such terms used in the prior art and the more specific use of the terms herein, the more specific meaning is meant.

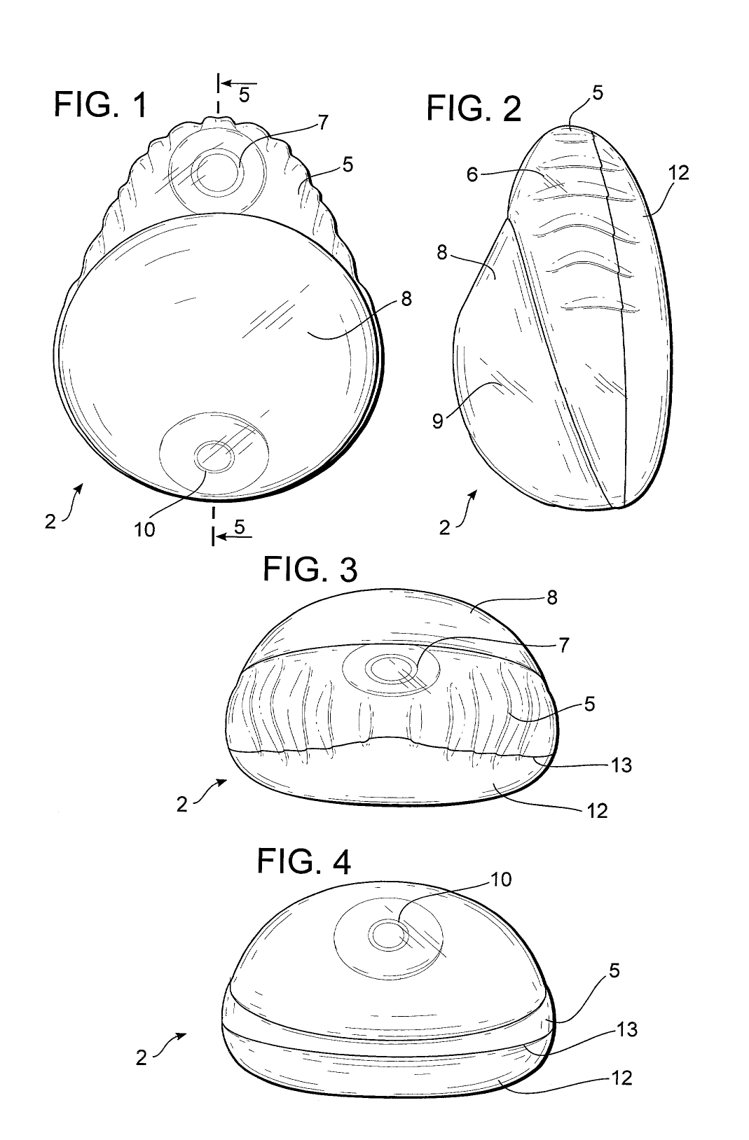

FIG. 1 illustrates a front view of the instant invention's immutably attached envelope(s) breast implant. As seen in FIG. 1 the breast prosthesis of the instant invention 2 is comprised of an immutably attached base envelope 5 and dome envelope 8. Each of said envelopes, (5, 8), embodies a valve (7, 10) formed as part of a wall to facilitate the introduction, containment or removal of fluid within a containable chamber of each said envelope. Practiced in one embodiment of the invention, the valve, 7, of the base envelope 5 is generally positioned at the apex of the substantially triangular shape based envelope 5, and valve 10 of the dome envelope 8 is generally placed in the lower region of envelope 8 to facilitate ease in introducing or removing fluid of the internal chambers of the envelopes (5, 8). FIG. 2 illustrates a side elevation view wherein the angular attachment of envelopes 5 and 8 may be better observed.

FIG. 2 shows the base envelope 5 shown in a vertical orientation with dome envelope 8 immutably sealed thereto and constituting the major projection of said prosthesis 2. The immutable sealing of envelopes may be facilitated by a cold chemical sealing means or heat treatment means well-known to those skilled in the art. Said bonding/attachment of envelopes can be facilitated by bonding either two distinct envelope structure means together, or by forming two distinct envelope structures by joining them together via a common (shared) wall. As is clearly disclosed and observed in FIG. 2, a “filled” internal chamber 6 of base envelope 5, and a “filled” internal chamber 9 of dome envelope 8 convincingly yield the shape of a female human breast. FIG. 2 also discloses the back portion, 12 of the prosthesis. Said back portion 12 is consistent in form and texture with base envelope 5 and dome envelope 8. The envelopes (5, 8)of the instant invention are comprised of a flexible and resilient medical grade silicone elastomer with surfaces making contact with breast tissue further embodying a contiguous dimpled or textured surface area. The manner of inducing a granulated, textured or “dimpled” surface to a medical grade silicone elastomer is well-known and practiced to those skilled in the art.

A typical, though non-limiting, method of manifesting the instant invention would call first for the angular bonding of dome envelope 8 to base envelope 5. In extensive testing it has been shown that an angular attachment of said envelope between 18° and 40° has been shown most effective to yield a female breast form having once filled said attached envelopes with saline, jet or other similarly intended fluid, though the invention is not to be limited by the outer bounds of said attachment, and may be so practiced. With the bonded prosthesis 2 then immersed into a chemical or other similarly intended composition known to induce a dimpling or otherwise textured surface to the outside walls of said envelopes, and then withdrawn. Valves similar to, though not limited to ball valves, (7, 10) are then inserted into each of the envelopes. The location of said valves are those generally indicated for the dome envelope 8 indicated by valve 10 and the for base envelope 5 as indicated by valve 7 of FIG. 1.

FIG. 3 illustrates a top elevation view of the instant invention, looking downwardly from valve 7 to dome envelope 8. In FIG. 3, the dome envelope 8 is shown providing the primary projection surface of said prosthesis to having been immutably and angularly attached to base envelope 5. FIG. 3 additionally shows the approximate location of a back surface area of the prosthesis indicated as element 12. Element 13 indicates the generalized location where said back surface typically originates.

FIG. 4 illustrates a bottom elevation view of the instant invention, viewing the invention from valve 10 of dome envelope 8 looking towards the apex of the generally triangular shaped base envelope 5 (apex not shown).

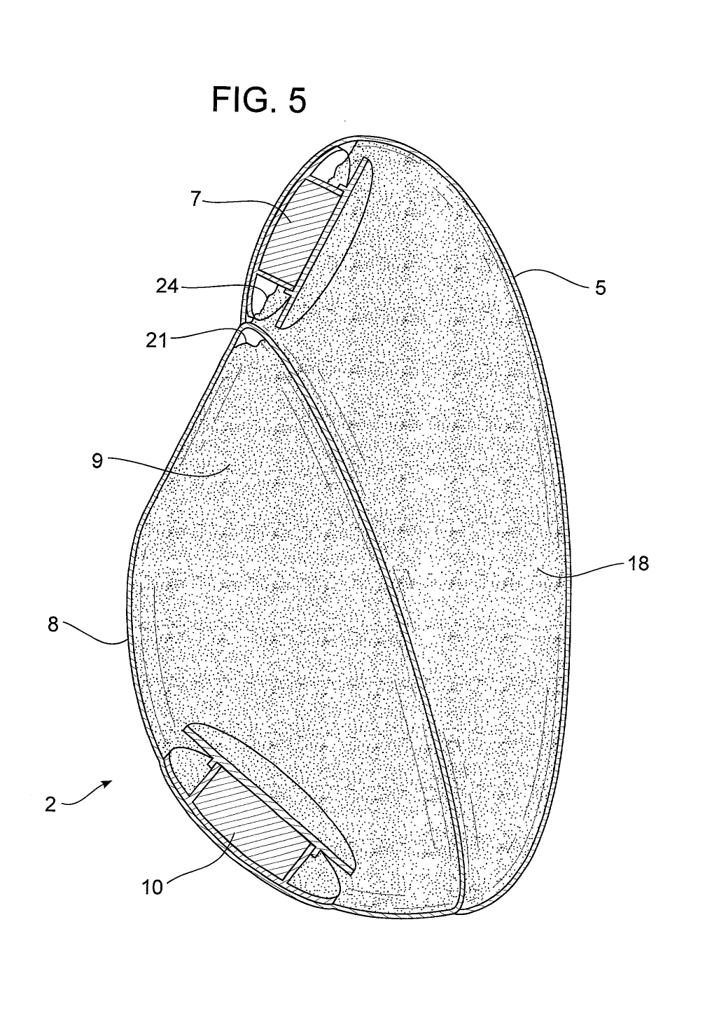

FIG. 5 is a cross-sectional view of instant invention wherein greater detail is provided with respect to the inner fluid containable chambers (9, 18) of dome envelope 8 and base envelope 5 respectively. Also shown in FIG. 5 is an illustration of saline or other similarly intended fluid residing within said chambers having been once so introduced following insertion of the device of the instant invention into a specifically formed breast cavity. Said fluid level indicated for the dome envelope 8 as 21 and fluid level for the base envelope 5 as 24.

It will be apparent to those skilled in the art that various modifications and variations can be made in the construction, configuration, and/or operation of the present invention without departing from the scope or spirit of the invention. For example, in the embodiments mentioned above, variations in the materials used to make each element of the invention may vary without departing from the scope of the invention. Thus, it is intended that the present invention cover the modifications and variations of the invention provided they come within the scope of the appended claims and their equivalents.

While the invention has been described with a certain degree of particularity, it is clear that many changes may be made in the details of construction and the arrangement of components without departing from the spirit and scope of this disclosure. It is understood that the invention is not limited to the embodiments set forth herein for purposes of exemplification, but is to be limited only by the scope of the attached claim or claims, including the full range of equivalency to which each element thereof is entitled.

What is claimed is:

1. A permanently implantable breast tissue prosthesis assembly comprising:
immutably attached base and dome envelopes each of which has a shell defining a inner fluid containable chambers and an outer textured surface to be in direct contact with breast tissue; and,
a valve formed as a part of a wall in each of said base and dome envelopes, said valve facilitating the introduction, containment or removal of fluid within the containable chamber of each envelope.

2. The breast prosthesis of claim 1 wherein said dome envelope is of substantially discoid shape.

3. The breast prosthesis of claim 1 wherein said base envelope is of substantially triangular shape.

4. The breast prosthesis of claim 1 wherein said dome and base envelopes are attached at an angle in a manner allowing differential unequal expansion to yield a human breast shape subsequent to introducing fluid to the containment chamber of each envelope.

5. The breast prosthesis of claim 1 wherein said dome and base envelopes are attached at an angle between 18° and 40°.

6. The breast prosthesis of claim 1 wherein dome and base envelopes are immutably attached via a chemical bonding means.

7. The breast prosthesis of claim 1 wherein dome and base envelopes are immutably attached via a heat bonding means.

8. The breast prosthesis of claim 1 wherein at least one valve formed as a part of a wall in each of said base and dome envelopes is a ball valve.

9. The breast prosthesis of claim 1 wherein said dome and base envelopes are bonded at an angle via a shared common wall.

10. A breast prosthesis assembly that is to be implanted into direct contact with tissue of said breast comprising:
a dome envelope element having a collapsed elastomeric first shell defining a first textured outer surface and enclosing a fillable first lumen, and first valve means integrated as a part of said first outer surface for introducing fluid into said first lumen;
a base element having a collapsed elastomeric second shell defining an second textured outer surface and enclosing a fillable second lumen, and second valve integrated as part of said second outer surface for introducing fluid into said second lumen;
means for immutably attaching a portion of said first outer surface to a portion of said second outer surface to form said assembly.

11. The breast prosthesis of claim 10 wherein said attached envelopes when filled with a fluid have the general shape of a human female breast.

12. A method for making a breast prosthesis assembly that is to be inserted into a human breast and implanted into direct contact with breast tissue, comprising the steps of:
forming at least two immutably and angularly attached implant elements, wherein each of said elements is defined as having an elastomeric envelope with a shell defining a contiguously textured outer surface, said envelopes having a given volume, and providing as a part of said outer surface a resealable valve means for variably introducing fluid into each said shell to achieve a desired cosmetic shape.

13. The method of claim 12, wherein said step of forming said implant elements further comprises the steps of:
inserting said immutably attached elements into said breast; and
filling said shell of each of said elements with said fluid, wherein the filling of each said shell can occur either pre-insertion or post-insertion.
